(19) United States
(12) Patent Application Publication
(10) Pub. No.: US 2010/0076352 A1
(43) Pub. Date: Mar. 25, 2010

(54) APPARATUS USING FOCUSED ULTRASOUND WAVE BY CONTROLLING ELECTRONIC SIGNALS AND USING METHOD THEREOF

(75) Inventors: Yong Tae Kim, Yuseong-gu (KR); Moon Jae Jho, Yuseong-gu (KR); Sung Soo Jung, Seo-gu (KR); Ho Chul Kim, Yuseong-gu (KR); Yong Hyeon Yun, Yuseong-gu (KR)

Correspondence Address: HISCOCK & BARCLAY, LLP, 2000 HSBC PLAZA, 100 Chestnut Street, ROCHESTER, NY, 14604-2404, US

(73) Assignee: Korea Research Institute of Standards and Science, Daejeon (KR)

(21) Appl. No.: 12/513,244

(22) PCT Filed: Dec. 4, 2006
(86) PCT No.: PCT/KR06/05178

§ 371 Date: May 1, 2009

(30) Foreign Application Priority Data
Nov. 22, 2006 (KR) 10-2006-0116052

(51) Int. Cl. A61N 7/00; A61B 8/00

(52) U.S. Cl. 601/2; 600/437

(57) ABSTRACT

The present invention relates to an extracorporeal High Intensity Focused Ultrasound (HIFU) necrosis apparatus through the control of an electronic signal, including oscillation elements for generating ultrasonic beams, an ultrasonic oscillator array having the oscillation elements fixed on a plane and oriented toward a life, delay circuits respectively connected to the oscillation elements for delaying ultrasonic oscillation by a delay time, and control means for controlling the delay time so that the ultrasonic beams are focused, and a method of employing the same. According to the present invention, waved surfaces with a variety of directions and curvatures can be formed using several oscillation elements and several delay circuits disposed on a plane, and a focus can be formed at any desired place. Accordingly, there are advantages in that installation is convenient, and a tissue, such as tumor, which is a target tissue, can be necrotized without damage to normal tissues necrosis. Further, there is no damage to normal tissues other than a target tissue. Accordingly, there are advantages in that a recovery speed of a patient can quicken, a symptom after recovery can be mitigated, and so on.

### TECHNICAL FIELD

The present invention relates, in general, to an extracorporeal High Intensity Focused Ultrasound (HIFU) necrosis apparatus through the control of an electronic signal, and more particularly, to a method of forming a focus using an electronic signal in a planar type array of an ultrasonic oscillator that is an ultrasonic source.

### BACKGROUND ART

In general, extracorporeal HIFU refers to ultrasonic wave that is obtained by focusing ultrasonic wave, generated outside a human body, on a target tissue (for example, tumor) within a life. Today, extracorporeal HIFU has been in the spotlight in the medical field because of its non-invasive characteristic.

A basic principle of HIFU, of selectively breaking tissues, such as tumor, by using a conventional ultrasonic wave is illustrated in FIG. 1. Tumor, etc. existing within a life becomes a target tissue 30, and an ultrasonic oscillator 10 is disposed outside a body so that the target tissue 30 is focused. Ultrasonic wave generated from the ultrasonic oscillator 10 is focused in the target tissue 30. Temperature rises at a tissue 40 of a point where a focus is brought in. When the temperature reaches a constant critical temperature (for example, 60 degrees Celsius or more), the tissue 40 on which a focus is formed, of the target tissue 30, is necrotized.

FIGS. 2 to 4 show states illustrating the ultrasonic oscillator 10, which is an extracorporeal ultrasonic source according to the principle of the conventional HIFU. If ultrasonic wave generated from the ultrasonic oscillator 10 is to be focused on the target tissue 30, the ultrasonic oscillator 10 that is geometrically concave, as shown in FIG. 2, is required. A piano-concave or plano-convex ultrasonic oscillator 10 can also be used, as shown in FIGS. 3 and 4.

The HIFU necrosis apparatus has been used to treat prostate tumor and hypertrophia in Korea since several years ago, and recently finds increasing application fields in various solid cancer treatments, including hepatoma. However, if a target tissue is eliminated using the conventional ultrasonic oscillator of the geometric form, there is a problem in that even normal tissues not having tumor are broken due to a limit accompanied by the geometric shape of the ultrasonic oscillator. A problem also arises in spatial limit for installation because the geometric curve of the ultrasonic oscillator is used.

### DISCLOSURE OF INVENTION

### Technical Problem

Accordingly, the present invention has been made keeping in mind the above problems occurring in the prior art, and an object of the present invention is to provide an extracorporeal HIFU necrosis apparatus through the control of an electronic signal and a method of using the same, in which an ultrasonic oscillator is constructed to have a planar shape not a geometric shape, so that convenience in installing the apparatus can be improved, an electrical signal can be controlled easily and precisely, and target tissues can be eliminated without damage to normal tissues.

### Technical Solution

The above object of the present invention is accomplished by an extracorporeal HIFU necrosis apparatus through the control of an electronic signal, including oscillation elements for generating ultrasonic beams; an ultrasonic oscillator array having the oscillation elements fixed on a plane and oriented toward a life; delay circuits respectively connected to the oscillation elements for delaying ultrasonic oscillation by a delay time; and control means for controlling the delay time so that the ultrasonic beams are focused.

The shape of the ultrasonic oscillator array 100 in which the oscillation elements 110 are arranged may be circular or square.

A length of a diameter or one side of the ultrasonic oscillator array 100 may be in the range of 15 to 30 cm.

The ultrasonic oscillation elements 110 may be ring-shaped, and a plurality of the ring-shaped ultrasonic oscillation elements 110 having different sizes may be disposed on a concentric circle.

The oscillation elements 110 may include piezoelectric material, a magnetostrictive transducer or a Capacitive Micromachined Ultrasonic Transducer (CMUT).

Furthermore, the above object of the present invention is accomplished by an extracorporeal HIFU necrosis apparatus through the control of an electronic signal, including an ultrasonic oscillator array 100 having ultrasonic oscillation elements 110 fixed on a plane and oriented toward a life; delay circuits 120 respectively connected to the oscillation elements 110 for delaying ultrasonic oscillation by a delay time; an ultrasonic oscillator including control means 150 for controlling the delay time so that the ultrasonic beams are focused; and 3-D image diagnosis means 170 for measuring the shape of a target tissue 30 within the life and outputting the measured shape as a 3-D image, wherein the control means 150 controls the delay time of the delay circuits 120 based on a positional data of the 3-D image in order to focus the ultrasonic beams on the target tissue 30.

The image diagnosis means 170 may include one of Magnetic Resonance Imaging (MRI), Computed Tomography (CT), and ultrasonic image diagnosis means.

Meanwhile, the extracorporeal HIFU necrosis apparatus may further include amplification means 140 for amplifying a signal applied to the ultrasonic oscillator array 100.

Furthermore, the extracorporeal HIFU necrosis apparatus may further include a multiplexer 130 for selectively applying a signal to the ultrasonic oscillator array 100.

The extracorporeal HIFU necrosis apparatus may further include necrosis design means 160 for designating a necrosis sequence based on the 3-D image between the image diagnosis means 170 and the control means 150. The control means 150 may control the delay time of the delay circuit 120 based on a necrosis sequence designated by the necrosis design means 160 in order to focus the ultrasonic waves on the target tissue 30.

Furthermore, the above object of the present invention is accomplished by a method of employing an extracorporeal HIFU necrosis apparatus through the control of an electronic signal, including a step (S100) of allowing 3-D image diagnosis means 170 to measure a shape of a target tissue 30 within a life and output a measured shape as a 3-D image; a step (S140) of allowing control means 150 to control delay time of each of delay circuits 120 connected to an ultrasonic oscillator array 100 based on a positional data of the 3-D image; a step (S180) of allowing the control means 150 to oscillate the ultrasonic oscillator array 100; and a step (S200) of making the target tissue 30 necrotized by focusing an oscillated ultrasonic wave on the target tissue 30 depending on a difference of the delay time.

Meanwhile, the method may further include a design step (S120) of specifying a necrosis sequence of the target tissue 30 and an intensity of an ultrasonic beam based on the 3-D image, after the step of outputting the 3-D image (S100).

Furthermore, the method may further include a step (S150) of amplifying a signal of the control means 150 and controlling ultrasonic sound power by controlling an amplification ratio, after the step (S140) of controlling the delay time.

Furthermore, the method may further include a step (S160) of selecting an oscillation element from which an ultrasonic beam is radiated and controlling a sound intensity I of an ultrasonic wave at a focus through the control of the amplification ratio, after the step (S150) of controlling the ultrasonic sound power.

The above object, and further objects and merits and characteristics of the present invention will become more apparent from the following detailed description and preferred embodiments taken in conjunction with the accompanying drawings.

### Advantageous Effects

According to the present invention, a waved surface having a variety of directions and curvatures can be formed using several oscillation elements and delay circuits existing on a plane. In other words, if an ultrasonic oscillator array having a predetermined shape upon fabrication is used, an installation position of an apparatus has to be changed, posing a limit to a focus brought in a desired place. If an electronic signal is used, however, there is an advantage in that a focus can be brought in any desired place without changing an installation position.

Accordingly, there is an advantage in that a tissue, such as tumor, which is a target tissue, can be necrotized without damage to normal tissues. Furthermore, if an extracorporeal HIFU necrosis apparatus through the control of an electronic signal according to the present invention is employed, there are advantages in that the recovery of a patient can quicken, a symptom after recovery can be mitigated, and so on because there is no damage to normal tissues other than target tissues.

### DESCRIPTION OF THE DRAWINGS

FIG. 1 shows a state illustrating the basic principle of a HIFU using a conventional extracorporeal HIFU,

FIGS. 2 to 4 show states illustrating an ultrasonic oscillator, which is an extracorporeal ultrasonic source according to the principle of the conventional HIFU,

FIG. 5 shows a state illustrating a basic principle of HIFU using an extracorporeal HIFU necrosis apparatus through the control of an electronic signal according to the present invention,

FIG. 6 is a front view illustrating a state where an oscillation element constituting a circular planar ultrasonic oscillator array, of the present invention, is disposed in matrix form,

FIG. 7 is a front view illustrating a state where a ring-shaped oscillation element constituting a circular planar ultrasonic oscillator array, of the present invention, is disposed on a concentric circle,

FIG. 8 is a front view of an oscillation element constituting a square planar ultrasonic oscillator array, of the present invention, is disposed,

FIG. 9 shows a system configuration of an extracorporeal HIFU necrosis apparatus through the control of an electronic signal according to the present invention,

FIG. 10 illustrates a radiation state of an ultrasonic beam having a short focus distance by employing time delay of the present invention,

FIG. 11 illustrates a radiation state of an ultrasonic beam having a long focus distance by employing time delay of the present invention,

FIG. 12 illustrates a radiation state of an ultrasonic beam that is set to face the front by employing time delay of the present invention,

FIG. 13 illustrates a radiation state of an ultrasonic beam having a focus downward by employing time delay of the present invention,

FIG. 14 illustrates a radiation state of an ultrasonic beam having a focus upward by employing time delay of the present invention,

FIG. 15 illustrates a radiation state of an ultrasonic beam whose focus distance and direction are controlled, and

FIG. 16 is a flowchart illustrating a method of employing the extracorporeal HIFU necrosis apparatus of the present invention.

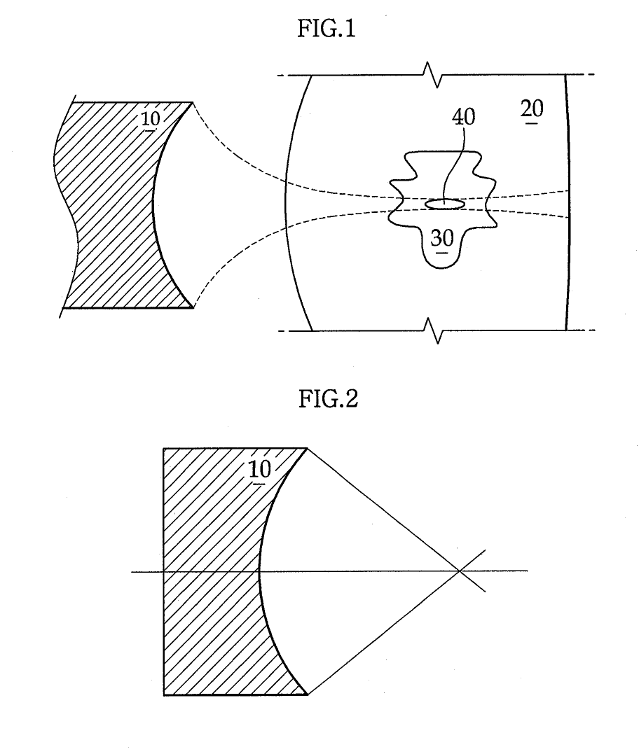

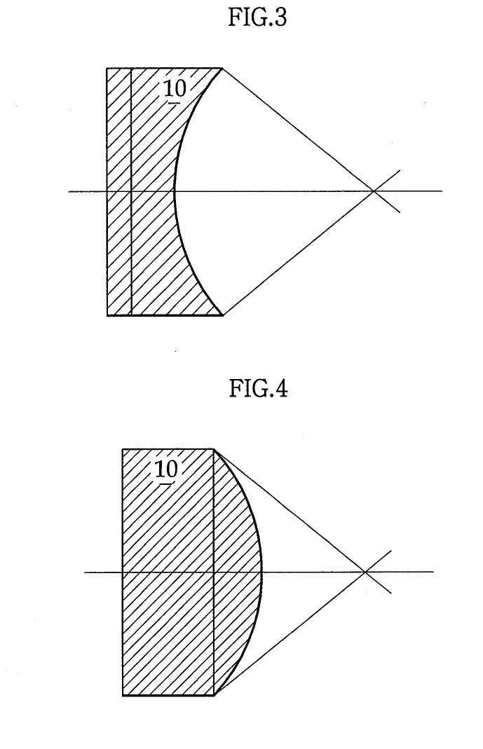

### BEST MODE FOR CARRYING OUT THE INVENTION

The present invention will now be described in detail in connection with preferred embodiments with reference to the accompanying drawings.

FIG. 5 schematically illustrates the principle of HIFU using a planar ultrasonic oscillator array 100, which is controlled in response to an electrical signal, according to the present invention.

The extracorporeal HIFU necrosis apparatus through the control of an electronic signal according to the present invention has the same basic principle as that of the conventional geometrical ultrasonic oscillator array 10 in that an ultrasonic beam is focused on a target tissue 30 in order to induce a temperature rise of a critical temperature (for example, 60 degrees Celsius) or more, and the target tissue 30 is necrotized. However, the extracorporeal HIFU necrosis apparatus of the present invention differs from the conventional geometrical ultrasonic oscillator array 10 in that an ultrasonic oscillator array 100 is formed on a plane. The planar ultrasonic oscillator array 100 has several ultrasonic oscillation elements 110 mounted therein. Ultrasonic waves are radiated from the respective ultrasonic oscillation elements 110 with a time lag and are then focused on the target tissue 30. As the ultrasonic waves are radiated with a time lag, a focus can be formed in the same manner as that a focus is formed by the geometric type ultrasonic oscillator.

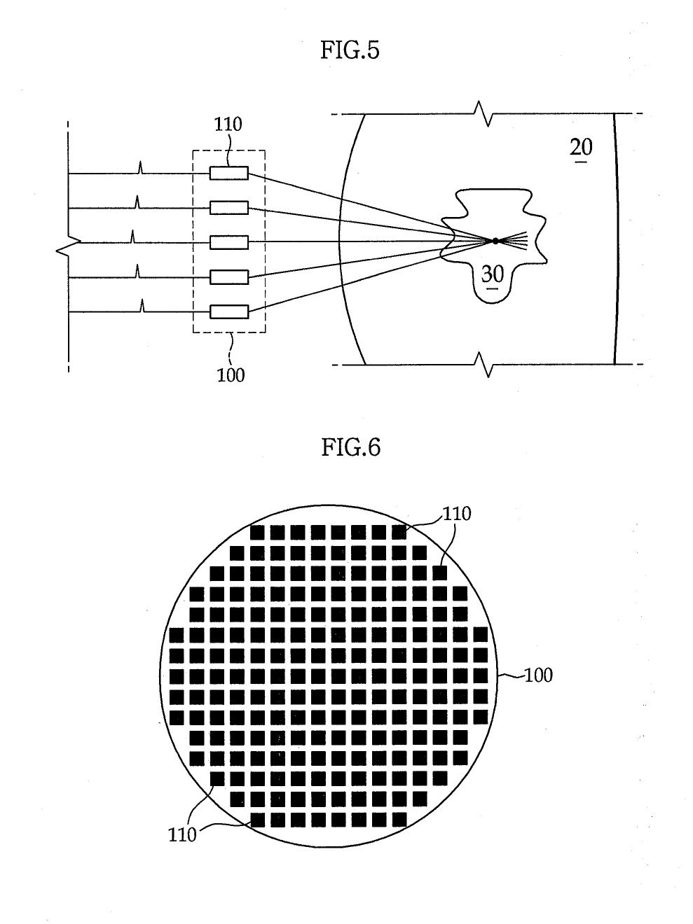

The front shape of the planar ultrasonic oscillator array 100 in which the oscillation elements 110 are arranged may be preferably isotropic, circular or square, as shown in FIGS. 6, 7 and 8. This is because it is efficient that the ultrasonic oscillator array 100 is isotropic in order to obtain the temperature rise effect within a short time by allowing a greater amount of the ultrasonic beam to reach a portion of the target tissue 30 in which a focus will be brought.

Furthermore, when considering the size of the target tissue 30 (for example, tumor) to be eliminated, the size of a life, etc., the length of a diameter or one side of the planar ultrasonic oscillator array 100 may be preferably in the range of 15 to 30 cm. When the length of a diameter or one side of the planar ultrasonic oscillator array 100 is 15 cm or less, it is difficult to expect a sufficient temperature rise in the target tissue 30 during a predetermined time since the number of the ultrasonic oscillation elements 110 from which ultrasonic waves are generated is less. When the length of a diameter or one side of the planar ultrasonic oscillator array 100 is 30 cm or more, there is a problem in that efficiency is low, such as that the intensity of an ultrasonic beam is weak and time taken for temperature to rise is long, because a focus distance is lengthened.

The oscillation elements 110 forming the planar ultrasonic oscillator array 100 can be disposed in matrix form, as shown in FIGS. 6 and 8.

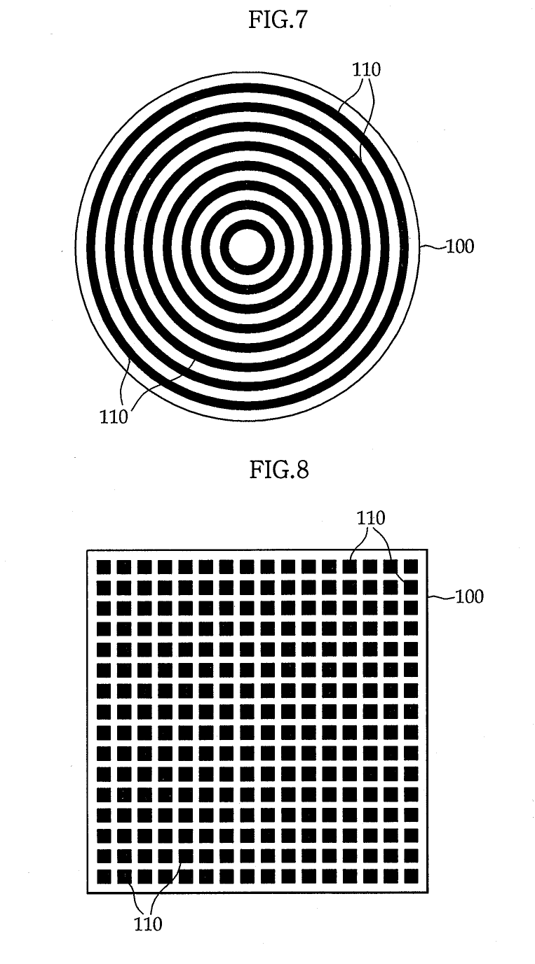

In this case, the oscillation elements 110 forming a circular ultrasonic oscillator array 100 can be disposed in a ring shape and the oscillation elements 110 with different radii can be disposed on a concentric circle, as shown in FIG. 7.

The shape of one of several ultrasonic oscillation elements 110 existing in the planar ultrasonic oscillator array 100 can be rectangular, circular, elliptical, fan-shaped, ring-shaped and so on as well as square shown in FIGS. 6, 7 and 8.

An oscillator that can be used as the ultrasonic oscillation element 110 can be formed from a piezoelectric material employing the piezoelectric effect, such as PZT-based material with a high electro-mechanical coupling coefficient, PMN-PT-based material with a good piezoelectric characteristic or LiNb-based material. A magnetostrictive transducer that varies according to a magnetization state can be used as the oscillator. A Capacitive Micromachined Ultrasonic Transducer (CMUT) can also be used as the oscillator. The CMUT is advantageous in that the same transducer can be fabricated and it can be directly applied to an electronic circuit because a semiconductor process is introduced.

As another embodiment of the extracorporeal HIFU necrosis apparatus through the control of an electronic signal, there is an extracorporeal HIFU necrosis apparatus through the control of an electronic signal, including the ultrasonic oscillator array 100 having the ultrasonic oscillation elements 110 fixed on a plane and oriented toward a life; the delay circuits 120 respectively connected to the oscillation elements 110 for delaying ultrasonic oscillation by a delay time; an ultrasonic oscillator including the control means 150 for controlling the delay time so that the ultrasonic beams are focused; and 3-dimensional (3-D) image diagnosis means 170 for measuring the shape of the target tissue 30 within the life and outputting the measured shape as a 3-D image. The control means 150 controls the delay time of the delay circuit 120 based on a positional data of the 3-D image in order to focus the ultrasonic beams on the target tissue 30.

It is preferred that the image diagnosis means 170 generally employ Magnetic Resonance Imaging (MRI), Computed Tomography (CT), ultrasonic image diagnosis means or the like.

It is preferred that amplification means 140 for amplifying a signal applied to the ultrasonic oscillator array 100 be further included.

It is preferred that a multiplexer 130 for selectively applying a signal to the ultrasonic oscillator array 100 be further included.

Furthermore, it is preferred that necrosis design means 160 for designating a necrosis sequence based on the 3-D image acquired by the 3-D image diagnosis means be further included between the image diagnosis means 170 and the control means 150.

The role and use method of each means, as a preferred embodiment of the extracorporeal HIFU necrosis apparatus through the control of an electronic signal, including the above means, is described below.

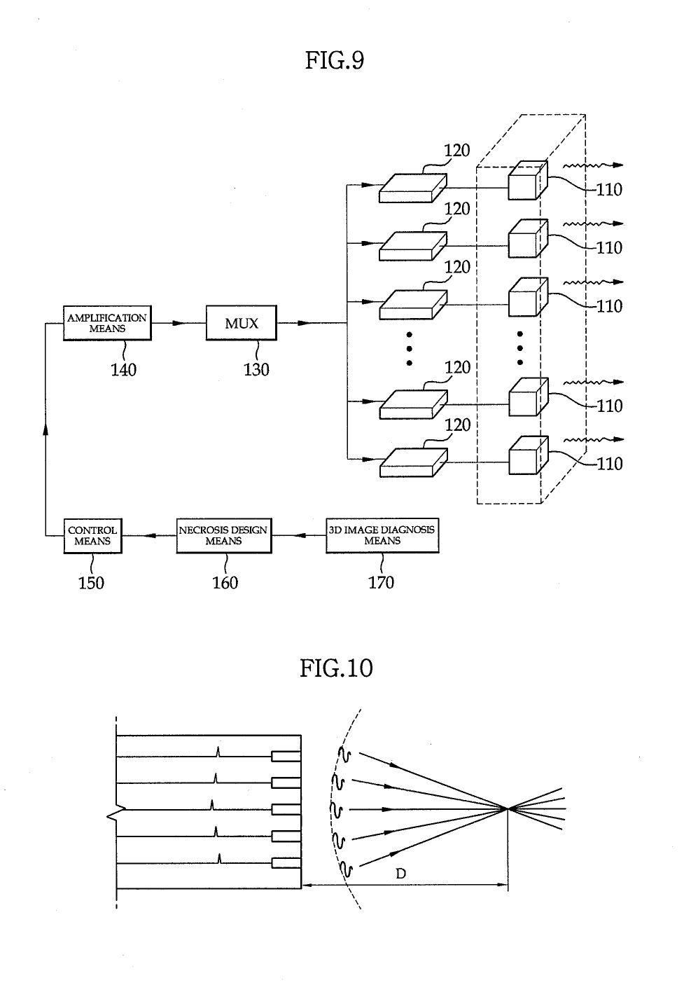

FIG. 9 shows a system configuration of the extracorporeal HIFU necrosis apparatus through the control of an electronic signal according to the present invention.

A 3-D image of the target tissue 30, which is acquired by the image diagnosis means 170, is sent to the necrosis design means 160.

The necrosis design means 160 establishes a plan for a sequence for breaking the target tissue 30, the intensity of an ultrasonic beam, etc. based on the 3-D image. It is intended not to give much burden on a life in which the target tissue 30 exists when the size of the target tissue 30 is small and is not simple in structure.

The control means 150 controls a delay time given to each delay circuit 120 based on a positional data of the 3-D image and the designed plan. Ultrasonic beams are radiated according to the delay time and are focused on the target tissue 30.

The amplification means 140 amplifies an electrical signal received from the control means 150. The signal amplified in the amplification means 140 is related to the intensity of the ultrasonic beams radiated from the oscillation elements 110. In other words, the output (that is, sound power P) of a desired ultrasonic beam can be controlled by controlling the amplification ratio (gain) of the amplification means 140. In this case, a total sound power P is decided depending on voltage Vᵢ applied to each of the oscillation elements 110 and radiation conductance Gᵢ of each oscillation element, and can be expressed in the following [Equation 1].

$$p = {\sum }_{i = 1}^{N} {G}_{i} \times {V}_{i}^{2} \qquad [ Equation 1 ]$$

The multiplexer 130 selects one of several oscillation elements 110 at a location where the ultrasonic beam has to be radiated, and sends the amplified electrical signal to one of the delay circuits 120 connected to the selected oscillation element 110. That is, it is not necessary to apply signals to the several delay circuits 120, respectively, using the multiplexer 130.

The plurality of delay circuits 120 are connected to the plurality of oscillation elements 110, respectively. The delay circuits 120 delay the signal received from the multiplexer 130 by a specific time, and induce the radiation of the ultrasonic beam. The plurality of delay circuits 120 cause a variety of delay times so that the radiated ultrasonic beams have various waved surfaces.

That is, by using the delay circuit 120 and the planar ultrasonic oscillator array 100, the same focus as that, which could be obtained in the conventional geometric ultrasonic oscillator array 10, can be obtained. Furthermore, the geometric ultrasonic oscillator array 100 is problematic in accurate control since spatial movement is required in the ultrasonic oscillator when the location of the focus is to be changed. However, in the extracorporeal focus type necrosis apparatus through the control of an electronic signal, a focus location can be changed accurately and easily through the use of only a delay time without spatial movement.

A method of controlling a focus location by employing a time delay of several ultrasonic oscillation elements 110 and several delay circuits 120 disposed in the ultrasonic oscillator array 100 is described in detail below.

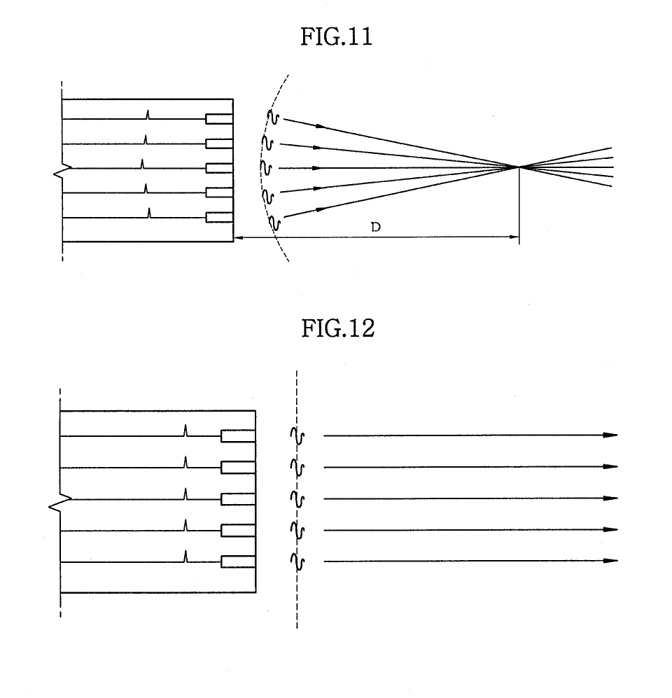

FIGS. 10 and 11 illustrate a method of controlling a focus distance of ultrasonic beams by employing a time delay.

FIG. 10 illustrates a method of forming a short focus distance. It is assumed that the illustrated oscillation elements 110 are sequentially assigned by S₁, S₂, S₃, S₄ and S₅ from the top to the bottom. In this state, in the case where ultrasonic waves are generated from the oscillation elements 110 having a symmetrical relationship around the oscillation elements (110) S₃, which is located at the center, that is, S₁ and S₅, and S₂ and S₄ at the same time, a waved surface of a curve can obtained. If a delay time distance between neighboring oscillation elements 110 (for example, S₁ and S₂) is set large, the curvature of the waved surface has a large value. Accordingly, a focus distance D is shortened.

FIG. 11 illustrates a method of forming a long focus distance. In the case where the delay time distance between neighboring oscillation elements 110 is set small compared with a case where the focus distance is short, a curvature has a small waved surface, and therefore the focus distance D is lengthened.

FIGS. 12 to 14 illustrate a method of controlling the direction of an ultrasonic beam by employing time delay.

Referring to FIG. 12, if ultrasonic waves are generated from the respective oscillation elements 110 at the same time, a waved surface is formed parallel to the plane ultrasonic oscillation array 100, and the beams orient toward the front.

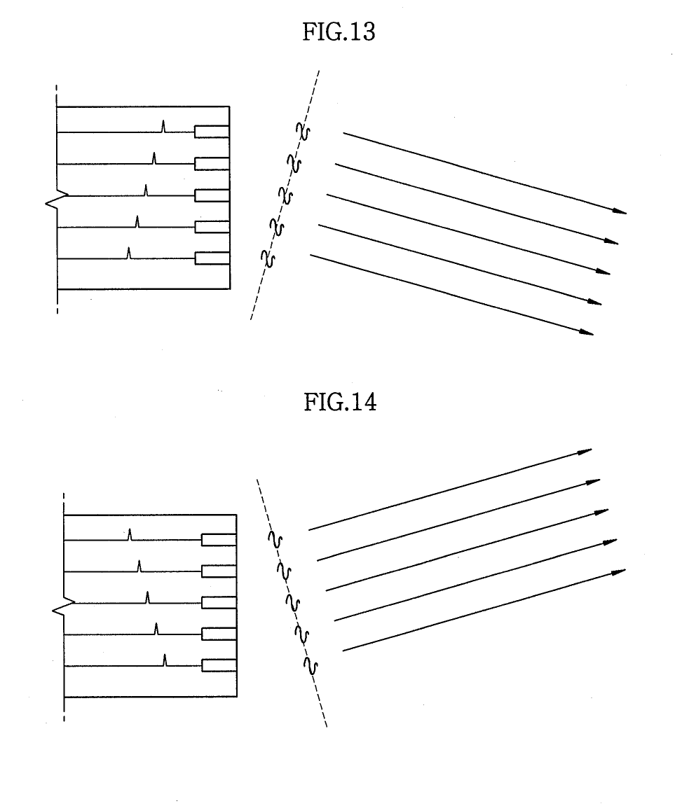

Referring to FIG. 13, if a beam is first generated from the oscillation element (110) S₁ and the beams S₂, S₃, S₄ and S₅ are then sequentially generated from the remaining oscillation elements 110 by a uniform time lag, waved surfaces are formed downward and the beams are oriented downward.

FIG. 14 illustrates a case where the oscillation sequence is reversed, and the waved surfaces are formed upward and the beams are oriented upward.

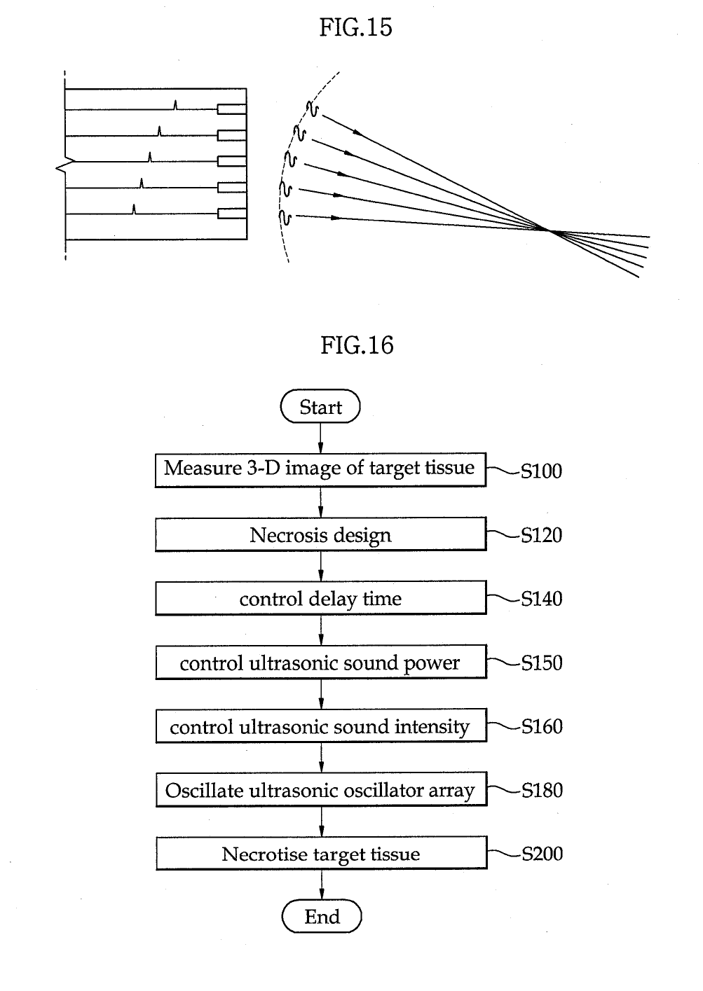

FIG. 15 is an example of a method of controlling both the focus distance and the direction of the ultrasonic beams described with reference to FIGS. 10 to 14. In the case where ultrasonic beams are radiated from the ultrasonic oscillation elements 110, which are symmetrical around the oscillation element 110 located at the center, and a delay time distance between S₁ and the remaining oscillation elements 110 is extended, a desired amount of a focus distance and direction can be obtained. That is, when a signal is applied with FIGS. 11 and 13 being coupled, a focus as shown in FIG. 15 can be obtained. By forming waved surfaces with various directions and curvatures by properly controlling a focus distance and the direction of a beam as described above, a variety of focuses can be formed. Therefore, a tissue of the target tissue 30 can be necrotized without damage to normal tissues.

Furthermore, by selecting an oscillation element through the multiplexer 130 and controlling the amplification ratio (gain) of the amplification means 140, a sound intensity I at the focus can be controlled.

At this time, the sound intensity I can be expressed in the following [Equation 2].

$$I = \frac{1}{A} \cdot {\sum }_{i = 1}^{{N}_{s}} {G}_{i} \times {V}_{i}^{2} x {e}^{- 2 α {d}_{i}} \qquad [ Equation 2 ]$$

where, dᵢ is a distance from each element to a focus distance, α is sound attenuation in a medium, A is a beam cross section in a focus, and Vᵢ is voltage applied to each oscillation element. Nₛ is the number of oscillation elements, which are selected by the multiplexer 130.

FIG. 16 illustrates a method of employing the extracorporeal HIFU necrosis apparatus through the control of an electronic signal according to a preferred embodiment.

The method includes a step (S100) of allowing the 3-D image diagnosis means 170 to measure the shape of the target tissue 30 within a life and output a measured shape as a 3-D image, a step (S140) of allowing the control means 150 to control delay time of each of the delay circuits 120 connected to the ultrasonic oscillator array 100 based on a positional data of the 3-D image, a step (S180) of allowing the control means 150 to oscillate the ultrasonic oscillator array 100, and a step (S200) of causing the target tissue 30 to be necrotized by focusing the oscillated ultrasonic wave on the target tissue 30 depending on a difference of the delay time.

It is preferred that after the 3-D image output step (S100), a design step (S120) of designating a necrosis sequence of the target tissue 30 and the intensity of an ultrasonic beam based on the 3-D image be further included. This is because a plan for the intensity of ultrasonic beams, a focus sequence, etc. for making a tissue necrotized when the target tissue 30 has a complicated structure or shape is required as mentioned earlier.

It is preferred that a step (S150) of amplifying signal of the control means 150 and controlling ultrasonic sound power by controlling an amplification ratio be further included, after the step (S140) of controlling the delay time. The ultrasonic sound power can be controlled by controlling the parameter values in [Equation 1], as mentioned earlier. This is because the ultrasonic sound power is concerned with the intensity of the ultrasonic beams radiated from the ultrasonic oscillation elements 110. In the method of amplifying the signal, it would be economical to amplify the signal at the control means 150 before the signal reaches the delay circuit 120 in terms of the manufacturing cost. However, a method of amplifying the signal at the control means 150 after the signal reaches the delay circuit 120, that is, when the signal reaches from the delay circuit 120 to the oscillation elements 110 may be used.

Furthermore, it is preferred that a step (S160) of selecting an oscillation element from which an ultrasonic beam is radiated and controlling the sound intensity I of an ultrasonic wave at a focus through the control of the amplification ratio be further includes, after the step (S150) of controlling the ultrasonic sound power. It is possible to predict the sound intensity at a point at which the focus of an ultrasonic beam is formed based on the above-mentioned [Equation 2] by controlling the sound power according to the step (S140) of controlling the ultrasonic sound power and selecting the oscillation element 110 from which the ultrasonic beam is radiated (in this case, the number of selected oscillation elements is indicated by Nₛ).

As described above, an optimal state for making the target tissue 30 necrotized within a life can be used safely for necrosis of the target tissue 30 without damage to normal tissues by predicting and controlling a focus location of an ultrasonic beam, the sound power P, and the sound intensity I at the focus.

### INDUSTRIAL APPLICABILITY

As described above, according to the present invention, waved surfaces with a variety of directions and curvatures can be formed using several oscillation elements and several delay circuits disposed on a plane. It is therefore possible to form a focus at any desired place without change of an installation position of an ultrasonic oscillator array. Furthermore, a tissue, such as tumor, which is a target tissue, can be necrotized without damage to normal tissues necrosis.

Further, if an extracorporeal HIFU necrosis apparatus through the control of an electronic signal according to the present invention is used, there is no damage to normal tissues other than a target tissue. Accordingly, there are advantages in that a recovery speed of a patient can quicken, a symptom after recovery can be mitigated, and so on.

Although the specific embodiments of the present invention have been disclosed for illustrative purposes, those skilled in the art will appreciate that various modifications, additions and substitutions are possible, without departing from the scope and spirit of the invention as disclosed in the accompanying claims.

1. An extracorporeal High Intensity Focused Ultrasound (HIFU) necrosis apparatus through the control of an electronic signal, comprising:
Oscillation elements 110 for generating ultrasonic beams;
an ultrasonic oscillator array 100 having the oscillation elements 110 fixed on a plane and oriented toward a life;
delay circuits 120 respectively connected to the oscillation elements 110 for delaying ultrasonic oscillation by a delay time; and
control means 150 for controlling the delay time so that the ultrasonic beams are focused.

2. The extracorporeal HIFU necrosis apparatus of claim 1, wherein the shape of the ultrasonic oscillator array 100 in which the oscillation elements 110 are arranged is circular or square.

3. The extracorporeal HIFU necrosis apparatus of claim 2, wherein a length of a diameter or one side of the ultrasonic oscillator array 100 is in the range of 15 to 30 cm.

4. The extracorporeal HIFU necrosis apparatus of claim 1, wherein:
the ultrasonic oscillation elements 110 are ring-shaped, and
a plurality of the ring-shaped ultrasonic oscillation elements 110 having different sizes are disposed on a concentric circle.

5. The extracorporeal HIFU necrosis apparatus of claim 1, wherein the oscillation elements 110 include piezoelectric material, a magnetostrictive transducer or a Capacitive Micromachined Ultrasonic Transducer(CMUT).

6. An extracorporeal HIFU necrosis apparatus through the control of an electronic signal, comprising:
an ultrasonic oscillator array 100 having ultrasonic oscillation elements 110 fixed on a plane and oriented a life;
delay circuits 120 respectively connected to the oscillation elements 110 for delaying ultrasonic oscillation by a delay time;
an ultrasonic oscillator including control means 150 for controlling the delay time so that the ultrasonic beams are focused; and
3-D image diagnosis means 170 for measuring the shape of a target tissue 30 within the life and outputting the measured shape as a 3-D image,
wherein the control means 150 controls the delay time of the delay circuits 120 based on a positional data of the 3-D image in order to focus the ultrasonic beams on the target tissue 30.

7. The extracorporeal HIFU necrosis apparatus of claim 6, wherein the 3-D image diagnosis means 170 includes one of Magnetic Resonance Imaging (MRI), Computed Tomography (CT), and ultrasonic image diagnosis means.

8. The extracorporeal HIFU necrosis apparatus of claim 6, further comprising amplification means 140 for amplifying a signal applied to the ultrasonic oscillator array 100.

9. The extracorporeal HIFU necrosis apparatus of claim 6, further comprising a multiplexer 130 for selectively applying a signal to the ultrasonic oscillator array 100.

10. The extracorporeal HIFU necrosis apparatus of claim 6, further comprising necrosis design means 160 for designating a necrosis sequence based on the 3-D image between the 3-D image diagnosis means 170 and the control means 150,
wherein the control means 150 controls the delay time of the delay circuit 120 based on the necrosis sequence designated by the necrosis design means 160 in order to focus the ultrasonic beams on the target tissue 30.

11. A method of employing an extracorporeal HIFU necrosis apparatus through the control of an electronic signal, the method comprising:
a step (S100) of allowing 3-D image diagnosis means 170 to measure a shape of a target tissue 30 within a life and output a measured shape as a 3-D image;
a step (S140) of allowing control means 150 to control delay time of each of delay circuits 120 connected to an ultrasonic oscillator array 100 based on a positional data of the 3-D image;
a step (S180) of allowing the control means 150 to oscillate the ultrasonic oscillator array 100; and
a step (S200) of making the target tissue 30 necrotized by focusing an oscillated ultrasonic wave on the target tissue 30 depending on a difference of the delay time.

12. The method of claim 11, further comprising a design step (S120) of specifying a necrosis sequence of the target tissue 30 and an intensity of an ultrasonic beam based on the 3-D image, after the step of outputting the 3-D image (S100).

13. The method of claim 11, wherein the step (S140) of controlling the delay time further includes a step (S150) of amplifying a signal of the control means 150 and controlling ultrasonic sound power by controlling the amplification ratio.

14. The method of claim 13, further comprising a step (S160) of selecting an oscillation element from which an ultrasonic beam is radiated and controlling a sound intensity I of an ultrasonic wave at a focus through the control of the amplification ratio, after the step (S150) of controlling the ultrasonic sound power.
